(19) United States
(12) Patent Application Publication
(10) Pub. No.: US 2024/0008991 A1
(43) Pub. Date: Jan. 11, 2024

(54) PROSTHETIC HIP REPLACEMENT ASSEMBLY WITH NEW AND IMPROVED PROSTHETIC BALL

(71) Applicant: JAMES STUART MELVIN, WASHINGTON, DC (US)

(72) Inventor: JAMES STUART MELVIN, WASHINGTON, DC (US)

(21) Appl. No.: 17/862,251

(22) Filed: Jul. 11, 2022

(51) Int. Cl. A61F 2/34

(52) U.S. Cl. CPC A61F 2/34; A61F 2310/00029; A61F 2310/00179; A61F 2002/30604

(57) ABSTRACT

A prosthetic hip replacement assembly comprises a femoral stem, a femoral head adapted to be fixedly secured to the femoral stem, an acetabular shell adapted to be inserted into a surgically prepared acetabulum, and a liner fixedly secured within the acetabular shell and within which the femoral head is adapted to fully articulate. The femoral head comprises a ball, a highly porous coating, and a plastic coating formed upon the highly porous coating such that the plastic coating can infiltrate the pores of the highly porous coating whereby the femoral head comprises a monobloc structure comprising the ball, the highly porous coating, and the plastic coating. In an alternative embodiment, the liner can be eliminated.

### FIELD OF THE INVENTION

The present invention relates generally to the field of prosthetic joints, and more particularly to a new and improved prosthetic hip replacement assembly, having a new and improved prosthetic ball component, which is to be utilized within hip replacement surgery.

### BACKGROUND OF THE INVENTION

Hip replacement is a commonly performed and very successful surgical procedure to rectify the pain and discomfort of hip arthritis. The most commonly utilized hip replacement assembly usually comprises four distinct components. As can best be appreciated from FIG. 1, a conventional hip replacement assembly is disclosed and is generally indicated by the reference character 10. More particularly, the hip replacement assembly 10 is seen to comprise a highly porous coated acetabular shell 12 which is fabricated as a metal cup having a highly porous coating formed thereon and which is adapted to be inserted into a surgically prepared acetabulum, not shown. In this manner, once the highly porous coated acetabular shell 12 is inserted or implanted into the surgically prepared acetabulum, bone growth can occur organically and interface with the micropores of the highly porous coated acetabular shell 12. A plastic liner 14, fabricated from a suitable thermoplastic material, such as, for example, polyethylene, is then inserted into and fixedly secured within the metal cup of the acetabular shell 12. A metal stem 16, that is, a femoral stem, is adapted to have its distal end fixedly inserted into a surgically prepared femur, while the opposite proximal end of the metal stem 16 is provided with an integrally formed trunnion or male morse taper 18. A metal or ceramic ball 20, which includes a female more taper 22, is rigidly connected to the proximal end of the femoral stem 16 as a result of the mating of the male and female morse tapers 18,22, such as for example, by means of an interference fit, or the like. This entire assembly is representative of a traditional modular hip replacement assembly.

Another conventional hip replacement assembly is disclosed within FIG. 2 and is generally indicated by the reference character 30. More particularly, the hip replacement assembly 30 is likewise seen to comprise a highly porous coated acetabular shell 32 which is fabricated as a metal cup 34 having a highly porous coating 36 formed thereon and which is adapted to be inserted into a surgically prepared acetabulum, not shown. However, unlike the conventional hip replacement assembly 10, as illustrated within FIG. 1, the conventional hip replacement assembly 30 does not include a liner 14. In lieu thereof, the internal surface of the metal cup 34 of the shell 32 is highly polished so as to receive a metal ball 38 which is adapted to be fixedly secured upon the proximal end of a femoral stem 40 by means of male and female morse tapers as has been described in connection with the hip replacement assembly 10 as illustrated within FIG. 1. This entire assembly is also representative of a traditional modular hip replacement assembly.

Recently, a dual mobility hip replacement construct or assembly has been developed with the objective of effectively providing the ball or femoral head, fixedly connected to the proximal end of the femoral stem, with a larger overall ball head size or diameter so as to substantially decrease the risk of dislocation of the ball or femoral head from the acetabular shell and liner. As can best be appreciated from FIG. 3, a conventional dual mobility hip replacement construct or assembly is disclosed and is generally indicated by the reference character 50. More particularly, it is seen that the dual mobility hip replacement construct or assembly 50 is seen to comprise a highly porous coated acetabular shell 52 which is fabricated as a metal cup having a highly porous coating formed thereon and which is adapted to be inserted into a surgically prepared acetabulum, not shown.

As was the case with the single mobility hip replacement construct or assembly 10, once the highly porous coated acetabular shell 52 is inserted or implanted into the surgically prepared acetabulum, bone growth can occur organically and interface with the micro-pores of the highly porous coated acetabular shell 52. A metal liner 54, fabricated from a suitable metal material, is then inserted into and fixedly secured within the metal cup of the acetabular shell 52. A metal stem 56, that is, a femoral stem, is adapted to have its distal end fixedly inserted into a surgically prepared femur, while the opposite proximal end of the metal stem 56 is provided with an integrally formed trunnion or male morse taper 58. A metal or ceramic ball 60, which includes a female morse taper 62, is rigidly connected to the proximal end of the femoral stem 56 as a result of the mating of the male and female morse tapers 58,62, such as for example, by means of an interference fit, or the like. In addition, a plastic ball 64, which may be fabricated from a suitable thermoplastic material, such as, for example, polyethylene, and which has a diametrical extent that is substantially greater or larger than that of the metal or ceramic ball 60, is snap-fitted over the metal or ceramic ball 60. It is to be noted that the metal or ceramic ball 60 is capable of fully pivoting or articulating within the larger plastic ball 64, and still further, the larger plastic ball 64 is, in turn, capable of fully pivoting or articulating within the metal acetabular liner 64, such pivotal or articulated movements providing the hip replacement construct or assembly with its dual mobility characteristics.

Unfortunately, it has been discovered that this dual mobility hip replacement construct or assembly 50 has led to some structural deficiencies which has caused the dual mobility hip replacement construct or assembly 50 to structurally fail, thereby causing significant problems for patients into which the dual mobility hip replacement construct or assembly 50 has been inserted or implanted. More particularly, there have been instances in which “intra-prosthetic” dislocation has occurred, that is, dislocation or separation of the smaller metal or ceramic ball 60 from the larger plastic ball 64. In addition, assembly of the smaller metal or ceramic ball 60 with the larger plastic ball 64 is required to be performed during the hip replacement surgical procedure, thereby requiring specialized equipment to be used during the surgical procedure which obviously requires the expenditure of additional time for completion of the surgical procedure. Still further, the metal composition utilized to fabricate the femoral stem 56 normally differs from the metal composition utilized to fabricate the smaller metal ball 60, or if the smaller metal ball 60 is fabricated from a suitable ceramic material, then obviously there is a difference in materials. For example, femoral stems are conventionally fabricated from a cobalt-chrome composition, stainless steel, tantalum, or titanium, while the smaller metal ball 60 may be fabricated from a cobalt-chrome composition or a ceramic material, it being noted that cobalt-chrome compositions can vary in their percentage content of either one of the cobalt and chrome components. In any case, these differences in materials comprising the fabrication of the femoral stem 56 and the smaller ball 60 can lead to galvanic corrosion or trunnionosis which is characterized as a total hip replacement arthroplasty failure.

Various prosthetic hip replacement assemblies have been developed over the years in order to address the foregoing and/or similar or related problems, however, none of them have been capable of doing so. In the interest of full disclosure, examples of such prior prosthetic hip replacement assemblies are disclosed within U.S. Pat. No. 11,285,007 which issued to Kavolus, II et al. on Mar. 29, 2022; United States Patent Application Publication 2017/0333192 which was published for Zhou et al. on Nov. 23, 2017; United States Patent Application Publication 2014/0094927 which was published for Weeden on Apr. 3, 2014; U.S. Pat. No. 8,357,205 which issued to Rahaman et al. on Jan. 22, 2013; United States Patent Application Publication 2006/025148 which was published for Bar-Ziv on Nov. 16, 2006; United States Patent Application Publication 2003/0120347 which was published for Steinberg on Jun. 26, 2003; United States Patent Application Publication 2002/0156536 which was published for Harris et al. on Oct. 24, 2002; U.S. Pat. No. 5,066,304 which issued to Crowninshield et al. on Nov. 19, 1991; U.S. Pat. No. 4,978,355 which issued to Frey et al. on Dec. 18, 1990; European Patent EP 0 768 066 which issued to Ernst et al. on Apr. 16, 1997; European Patent Application EP 1 582 182 which was published for Aux Epaules et al. on Oct. 5, 2005; Chinese Patent Application CN 105030378 which was published on Nov. 11, 2015; PCT Patent Application Publication WO 2016/183701 which was published for Zhu et al. on Nov. 24, 2016; PCT Patent Application Publication WO 2019/171158 which was published for Perez Nunez on Sep. 12, 2019; European Patent Application Publication EP 3 777 775 which was published for Melozzi on Feb. 17, 2021; and European Patent Application Publication EP 3 777 776 which was published for Melozzi on Feb. 17, 2021.

A need therefore exists in the art for a new and improved prosthetic hip replacement assembly. Another need exists in the art for a new and improved prosthetic hip replacement assembly which resolves the aforenoted problems. Still another need exists in the art for a new and improved prosthetic hip replacement assembly which resolves the aforenoted problems, such as, for example, substantially decreasing the risk of dislocation of the femoral head from the acetabular shell and liner. Yet another need exists in the art for a new and improved prosthetic hip replacement assembly which resolves the aforenoted problems, such as, for example, substantially decreasing the risk of any intra-prosthetic dislocation which has been experienced with dual-mobility hip replacement constructs or assemblies. Still yet another need exists in the art for a new and improved prosthetic hip replacement assembly which resolves the aforenoted problems, such as, for example, significantly reducing the time required to complete the prosthetic hip replacement surgical procedure. Yet still another need exists in the art for a new and improved prosthetic hip replacement assembly which resolves the aforenoted problems, such as, for example, galvanic corrosion which may occur when different, incompatible metals are utilized to fabricate the femoral stem and the femoral ball or head. An additional need exists in the art for a new and improved prosthetic hip replacement assembly which resolves the aforenoted problems, such as, for example, trunnionosis, which is characterized as a total hip replacement arthroplasty failure.

### OVERALL OBJECTIVES OF THE PRESENT INVENTION

An overall objective of the present invention is to provide a new and improved prosthetic hip replacement assembly. Another overall objective of the present invention is to provide a new and improved prosthetic hip replacement assembly which resolves the aforenoted problems. Still another overall objective of the present invention is to provide a new and improved prosthetic hip replacement assembly which resolves the aforenoted problems, such as, for example, substantially decreasing the risk of dislocation of the femoral head from the acetabular shell and liner. Yet another overall objective of the present invention is to provide a new and improved prosthetic hip replacement assembly which resolves the aforenoted problems, such as, for example, substantially decreasing the risk of any intra-prosthetic dislocation which has been experienced with dual-mobility hip replacement constructs or assemblies. Still yet another overall objective of the present invention is to provide a new and improved prosthetic hip replacement assembly which resolves the aforenoted problems, such as, for example, significantly reducing the time required to complete the prosthetic hip replacement surgical procedure. Yet still another overall objective of the present invention is to provide a new and improved prosthetic hip replacement assembly which resolves the aforenoted problems, such as, for example, galvanic corrosion which may occur when different, incompatible metals are utilized to fabricate the femoral stem and the femoral ball or head. An additional overall objective of the present invention is to provide a new and improved prosthetic hip replacement assembly which resolves the aforenoted problems, such as, for example, trunnionosis, which is characterized as a total prosthetic hip replacement arthroplasty failure.

### SUMMARY OF THE INVENTION

In accordance with the principles and teachings of the present invention, a new and improved prosthetic hip replacement assembly has been developed and is seen to comprise a highly porous coated acetabular shell which is fabricated as a metal cup having a highly porous coating formed thereon and which is adapted to be inserted into a surgically prepared acetabulum. In this manner, once the highly porous coated acetabular shell is inserted or implanted into the surgically prepared acetabulum, bone growth can occur organically and interface with the micropores of the highly porous coated acetabular shell. A metal liner, fabricated from a suitable metal and having a thickness dimension which is smaller than the conventional thermoplastic liner material, is then inserted into and fixedly secured within the metal cup of the acetabular shell. A metal stem, that is, a femoral stem, is adapted to have its distal end fixedly inserted into a surgically prepared femur, while the opposite proximal end of the metal stem is provided with an integrally formed trunnion or male morse taper.

A metal ball, or femoral head, which includes a female morse taper, is adapted to be rigidly connected to the proximal end of the femoral stem as a result of the mating of the male and female morse tapers, such as for example, by means of an interference fit, or the like. Both the metal ball, or femoral head, as well as the femoral stem may be fabricated from the same suitable cobalt-chromium compositions, stainless steel, tantalum, or titanium. Furthermore, in accordance with more specific principles and teachings of the present invention, the metal ball, which effectively defines the basic structure of the femoral head, has an open-cell, highly porous metallic coating formed thereon, and then the highly porous coating is, in turn, coated with a suitable thermoplastic coating which may comprise, for example, highly cross-linked polyethylene. Well-known highly porous metallic coatings, such as, for example, those manufactured by DEPUY and sold under the trademark GRIPTION®, or those manufactured by SMITH & NEPHEW and sold under the trademark STIKTITE®, have micropores within the range of 100-1,000 microns. Accordingly, the thermoplastic coating is able to completely infiltrate the highly porous coating such that the thermoplastic and highly porous coatings can effectively interdigitate with each other so as to be integrally connected together. In other words, it can be readily appreciated that the resulting structure of the femoral head effectively forms a structural monobloc comprising the internal metal ball, the highly porous metallic coating coated upon the internal metal ball, and the outer coating of highly cross-linked polyethylene (HCLPE) coated upon the highly porous metallic coating. This ball or femoral head is then capable of being disposed within the metal liner disposed within the highly porous coated acetabular shell whereby a highly stable, but fully articulating prosthetic hip replacement assembly has been constructed. In accordance with a second embodiment of the present invention, the liner may effectively be eliminated whereby the structural monobloc comprising the internal metal ball, the highly porous metallic coating coated upon the internal metal ball, and the outer coating of highly cross-linked polyethylene (HCLPE) coated upon the highly porous metallic coating, is disposed within a highly polished internal surface of the metal cup forming the support structure of the highly porous coated acetabular shell.

### BRIEF DESCRIPTION OF THE DRAWINGS

Various other features and attendant advantages of the present invention will be more fully appreciated from the following detailed description when considered in connection with the accompanying drawings in which like reference characters designate like or corresponding parts throughout the several views, and wherein:

FIG. 1 is an exploded view of a conventional PRIOR ART hip replacement assembly which includes a liner disposed within the metal cup of the acetabular shell;

FIG. 2 is a partially exploded view of a conventional PRIOR ART hip replacement assembly which does not have a liner disposed within the metal cup of the acetabular shell, and wherein the femoral head is disposed directly within the metallic cup of the acetabular head;

FIG. 3 is an exploded view of a conventional PRIOR ART dual mobility hip replacement assembly;

FIG. 4 is a partially exploded view of a first embodiment of a new and improved hip replacement assembly, which includes the new and improved femoral head or ball, as constructed in accordance with the principles and teachings of the present invention;

FIG. 5 is a schematic cross-sectional view of the new and improved femoral head or ball as illustrated within FIG. 4 and as constructed in accordance with the principles and teachings of the present invention;

FIG. 6 is a highly magnified view of the highly porous coating covering the metal ball or femoral head, as well as the acetabular shell; and

FIG. 7 is a partially exploded view of a second embodiment of a new and improved hip replacement assembly, which includes the new and improved femoral head or ball but eliminates the metal liner, as constructed in accordance with the principles and teachings of the present invention.

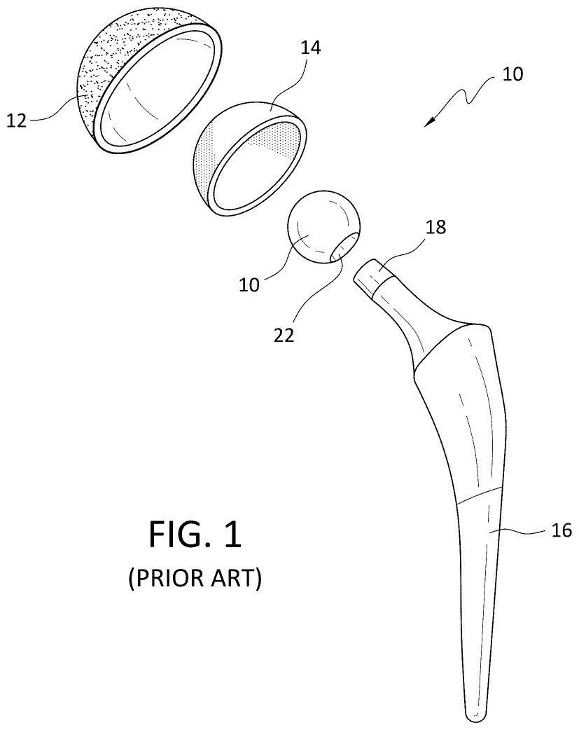

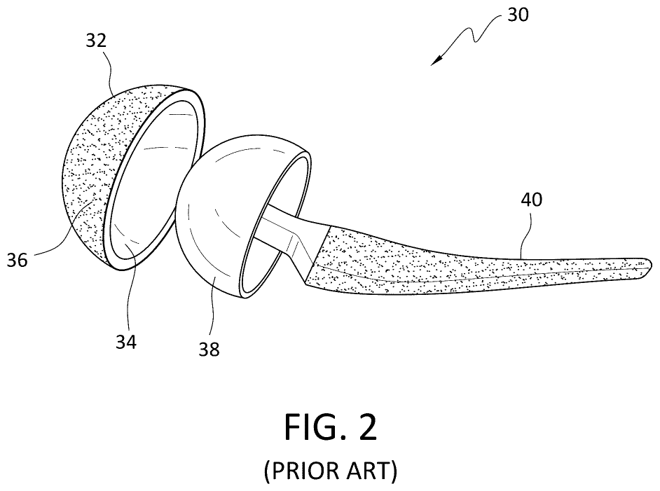

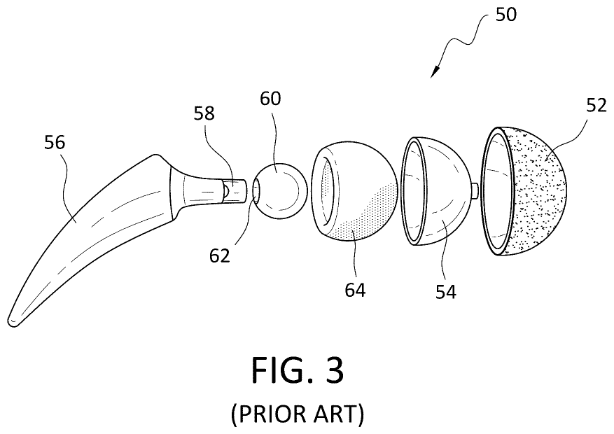

### DETAILED DESCRIPTION OF THE EMBODIMENTS

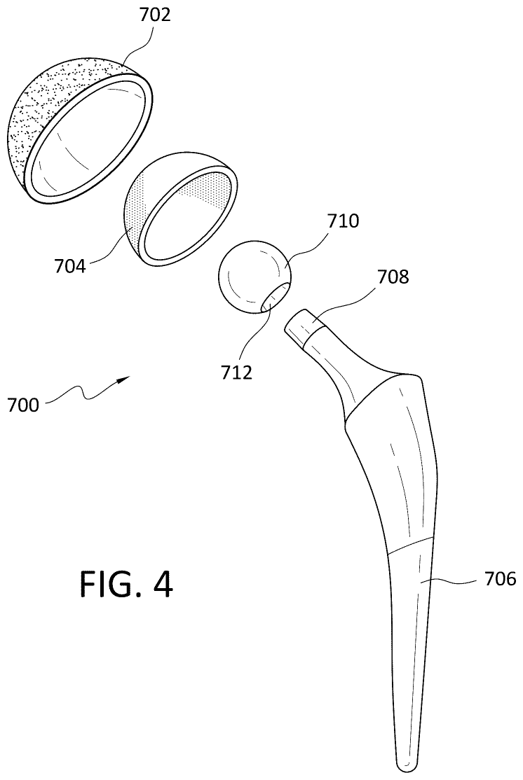

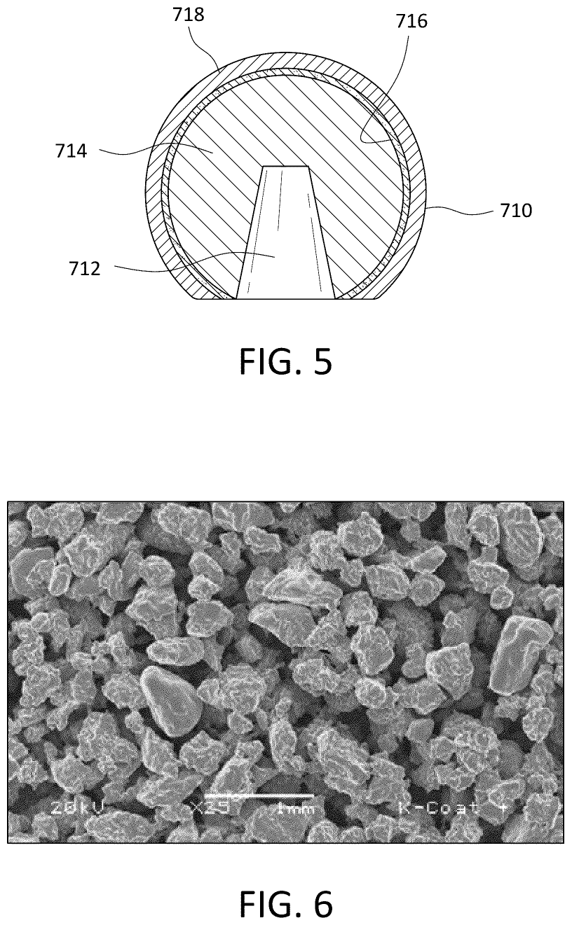

With reference now being made to FIGS. 4-6, and in accordance with the principles and teachings of the present invention, a first embodiment of a new and improved prosthetic hip replacement assembly has been developed and is disclosed by the reference character 700. More particularly, it is seen that the new and improved prosthetic hip replacement assembly 700 comprises an open-cell, highly porous metallically coated acetabular shell 702 which is fabricated as a metal cup having an open-cell highly porous metallic coating formed thereon and which is adapted to be inserted into a surgically prepared acetabulum. In this manner, once the highly porous coated acetabular shell 702 is inserted or implanted into the surgically prepared acetabulum, bone growth can occur organically and integrally interface with the micropores of the highly porous coated acetabular shell 702. A metal liner 704, fabricated from a suitable metal and having a thickness dimension which is smaller than that of conventional thermoplastic liners, is then inserted into and fixedly secured within the metal cup of the acetabular shell 702. A metal stem, that is, a femoral stem 706, is, in accordance with the principles and teachings of the present invention, fabricated from a suitable cobalt-chromium composition, stainless steel, tantalum, or titanium, and is adapted to have its distal end fixedly inserted into a surgically prepared femur, while the opposite proximal end of the metal stem is provided with an integrally formed trunnion or male morse taper 708. As can best be appreciated from FIGS. 4 and 5, a femoral head 710 includes a female morse taper 712 which is defined internally within a metal ball 714, and is adapted to be rigidly connected to the proximal end of the femoral stem 706 as a result of the mating of the male and female morse tapers 708,712, such as for example, by means of an interference fit, or the like. Both the metal ball 714 of the femoral head 710, as well as the femoral stem 706, may be fabricated from the same suitable cobalt-chromium composition, stainless steel, tantalum, or titanium.

Furthermore, in accordance with more specific principles and teachings of the present invention, and again, as can best be appreciated from FIG. 5, the femoral head 710 comprises a metal ball 714, which effectively defines the basic structure of the femoral head 710, an open-cell, highly porous metallic coating 716 formed thereon, and then the open-cell highly porous metallic coating 716 is, in turn, coated with a suitable thermoplastic coating 718 which may comprise, for example, highly cross-linked polyethylene (HCLPE). Well-known open-cell, highly porous metallic coatings, which may be utilized as the highly porous coating 716, can be selected from any well-known, commercially available highly porous coatings, such as, for example, those manufactured by DEPUY and sold under the trademark GRIPTION®, or those manufactured by SMITH & NEPHEW and sold under the trademark STIKTITE®. These highly porous coatings have pores within the range of 100-1,000 microns, and accordingly, the plastic coating 718 is able to completely infiltrate the highly porous coating 716 and be integrally attached thereto as a result of the thermoplastic and highly porous coatings effectively interdigitating with each other so as to be integrally connected together. In other words, it can be readily appreciated that the resulting structure of the femoral head effectively forms a structural monobloc comprising the internal metal ball, the open-cell, highly porous metallic coating coated upon the internal metal ball, and the outer coating of highly cross-linked polyethylene (HCLPE) coated upon the highly porous metallic coating. This femoral head 710 is then capable of being disposed within the metal liner 704 disposed within the highly porous coated acetabular shell 702, whereby a new and improved, highly stable, but fully articulating prosthetic hip replacement assembly has been constructed. It is to be lastly noted that the outer coating of highly cross-linked polyethylene (HCLPE) 718 can have a thickness dimension which is within the range of 2-18 mm.

With reference being made to FIG. 6, FIG. 6 is a highly magnified view of a typical open-cell, highly porous coating, such as those manufactured by DEPUY or SMITH & NEPHEW, as has been previously noted, wherein it is to be noted that while the average pore size of the open-cell, highly porous metallic coating may be 200 microns, the pore size can be within an optimal range of 100-1,000 microns for optimal bone growth, when such coatings are utilized as the coating upon the highly porous coated acetabular shell 702, as well as to promote the development of the monobloc femoral head 710 when the highly cross-linked polyethylene (HCLPE) coating 718 is deposited upon the highly porous coating 716 of the femoral head 710.

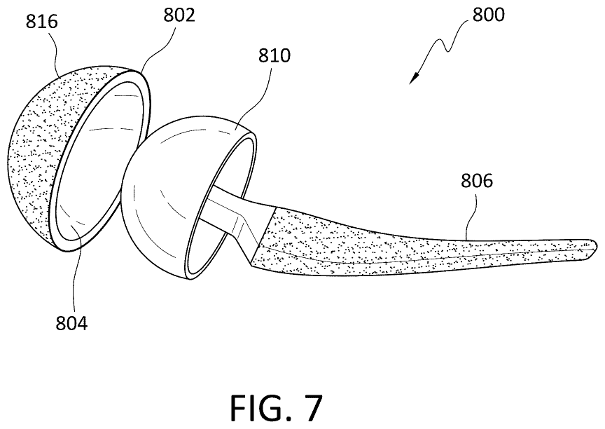

With reference lastly being made to FIG. 7, and in accordance with additional principles and teachings of the present invention, a second embodiment of a new and improved prosthetic hip replacement assembly has been developed and is disclosed by the reference character 800. It is to be noted that component parts of the second embodiment of the new and improved prosthetic hip assembly, that correspond to component parts of the first embodiment of the new and improved prosthetic hip assembly will be denoted by corresponding reference characters except that they will be within the 800 series. In addition, only the difference between the first and second embodiments of the new and improved prosthetic hip assembly will be discussed. More particularly, the only significant difference between the first and second embodiments of the new and improved prosthetic hip assemblies 700 and 800 resides in the fact that the liner 704 of the first embodiment of the new and improved prosthetic hip assembly 700 has effectively been eliminated wherein the new and improved monobloc ball assembly 810 is adapted to be disposed within the highly polished, internal surface 804 of the acetabular shell 802.

Obviously, many variations and modifications of the present invention are possible in light of the above teachings. For example, the femoral head, monobloc structure 510 can be fabricated so as to have various different head sizes having various different diametrical extents. In addition, and in a similar manner, the male and female morse tapers 508,512 may likewise be provided with various different sizes. It is therefore to be understood that within the scope of the appended claims, the present invention may be practiced otherwise than as specifically described herein.

What is claimed as new and desired to be protected by Letters Patent, is:

1. A prosthetic hip replacement assembly, comprising:
a femoral stem;
a femoral head adapted to be fixedly secured to said femoral stem;
an acetabular shell adapted to be inserted into a surgically prepared acetabulum; and
a liner fixedly secured within said acetabular shell and within which said femoral head is adapted to fully articulate;
wherein said femoral head comprises a ball, a highly porous coating formed upon said ball, and a thermoplastic coating formed upon said highly porous coating such that said thermoplastic coating can infiltrate said highly porous coating whereby said femoral head comprises a monobloc structure comprising said ball, said highly porous coating, and said thermoplastic coating.

2. The assembly as set forth in claim 1, wherein:
said femoral stem is fabricated from a material selected from the group comprising a cobalt-chromium composition, tantalum, stainless steel, and titanium.

3. The assembly as set forth in claim 1, wherein:
said ball of said femoral head is fabricated from a material selected from the group comprising a cobalt-chromium composition, tantalum, stainless steel, and titanium.

4. The assembly as set forth in claim 1, wherein:
both said femoral stem and said ball of said femoral head are fabricated from a material selected from the group comprising a cobalt-chromium composition, tantalum, stainless steel, and titanium.

5. The assembly as set forth in claim 1, wherein:
said femoral stem comprises a male morse taper; and
said ball of said femoral head comprises a female morse taper,
whereby when said male and female morse tapers are fixedly connected together, said femoral head is fixedly mounted upon said femoral stem.

6. The assembly as set forth in claim 1, wherein:
said liner of said assembly is fabricated from a suitable metal.

7. The assembly as set forth in claim 1, wherein:
said highly porous coating comprises an open-cell, highly porous metallic coating.

8. The assembly as set forth in claim 7, wherein:
said highly porous coating comprises an open-cell, highly porous metallic coating having pores which are within the range of 100-1000 microns.

9. The assembly as set forth in claim 1, wherein:
said thermoplastic coating comprises highly cross-linked polyethylene (HCLPE).

10. The assembly as set forth in claim 1, wherein:
said thermoplastic coating has a thickness dimension within the range of 2-18 mm.

11. A prosthetic hip replacement assembly, comprising:
a femoral stem;
a femoral head adapted to be fixedly secured to said femoral stem; and
an acetabular shell adapted to be inserted into a surgically prepared acetabulum and within which said femoral head is adapted to articulate;
wherein said femoral head comprises a ball, a highly porous coating formed upon said ball, and a thermoplastic coating formed upon said highly porous coating such that said thermoplastic coating can infiltrate said highly porous coating whereby said femoral head comprises a monobloc structure comprising said ball, said highly porous coating, and said thermoplastic coating.

12. The assembly as set forth in claim 11, wherein:
said femoral stem is fabricated from a material selected from the group comprising a cobalt-chromium composition, tantalum, stainless steel, and titanium.

13. The assembly as set forth in claim 11, wherein:
said ball of said femoral head is fabricated from a material selected from the group comprising a cobalt-chromium composition, tantalum, stainless steel, and titanium.

14. The assembly as set forth in claim 11, wherein:
both said femoral stem and said ball of said femoral head are fabricated from a material selected from the group comprising a cobalt-chromium composition, tantalum, stainless steel, and titanium.

15. The assembly as set forth in claim 11, wherein:
said femoral stem comprises a male morse taper; and
said ball of said femoral head comprises a female morse taper,
whereby when said male and female morse tapers are fixedly connected together, said femoral head is fixedly mounted upon said femoral stem.

16. The assembly as set forth in claim 11, wherein:
said acetabular shell comprises a highly polished internal surface fabricated from a suitable metal upon which said femoral head can fully articulate.

17. The assembly as set forth in claim 11, wherein:
said highly porous coating comprises an open-cell, highly porous metallic coating.

18. The assembly as set forth in claim 17, wherein:
said highly porous coating comprises an open-cell, highly porous metallic coating having pores which are within the range of 100-1000 microns.

19. The assembly as set forth in claim 11, wherein:
said thermoplastic coating comprises highly cross-linked polyethylene (HCLPE).

20. The assembly as set forth in claim 11, wherein:
said thermoplastic coating has a thickness dimension within the range of 2-18 mm.
